(19) United States
(12) Patent Application Publication
(10) Pub. No.: US 2024/0066487 A1
(43) Pub. Date: Feb. 29, 2024

(54) IMPROVED DEVICE FOR FACILITATING A CHEMICAL REACTION

(71) Applicants: Alyssa SCHROETER, Medina, OH (US); Todd SCHROETER, Medina, OH (US)

(72) Inventors: Alyssa SCHROETER, Medina, OH (US); Todd SCHROETER, Medina, OH (US)

(21) Appl. No.: 18/261,001

(22) PCT Filed: Jan. 13, 2022
(86) PCT No.: PCT/US2022/012282

§ 371 Date: Jul. 11, 2023

Related U.S. Application Data
(60) Provisional application No. 63/136,986, filed on Jan. 13, 2021.

(51) Int. Cl. B01J 7/02; C01B 11/02

(52) U.S. Cl. CPC B01J 7/02; C01B 11/024

(57) ABSTRACT

An improved device for facilitating a chemical reaction while submerged in a liquid catalyst includes an upper member, a lower member, and a dissolvable member with apertures disposed between and ultimately enclosed by said upper and lower members such that upper and lower chambers are formed having substantially equal volumes. The upper chamber may receive a dry sodium chlorite and the lower chamber may receive a dry acid or acid mixture.

### CROSS REFERENCES AND PRIORITIES

This application claims priority from U.S. Provisional Patent Application No. 63/136,986 filed 13 Jan. 2021, the teachings of which are incorporated by reference herein in their entirety.

### BACKGROUND

### Field of the Invention

This invention relates to an improved device for facilitating a chemical reaction, and more particularly, to a device and method for facilitating the generation of chlorine dioxide gas for release into air or water.

### Background of the Prior Art

Chlorine dioxide gas is a well-known disinfectant and deodorizing agent that can be generated as a gas for release into air or water. Chlorine dioxide gas is soluble and does not hydrolyze in water, but remains as a true gas in water. It is common to use sodium chlorite and an acid, both in dry form, combined with an aqueous solution to generate chlorine dioxide. The problem with conventional non-electrically powered chlorine dioxide gas generators using dry sodium chlorite and an acid has been the membrane shells forming the cavities that receive the dry sodium chlorite and acid. More specifically, prior art membranes are substantially impervious to liquid and have been designed to protect the dry internal components from moisture to promote shipping and handling of the device without activation.

Unfortunately, the water protective membranes have increased the reaction time required for completing the chlorine dioxide gas generation from the combining of the sodium chlorite and acid after exposure to water. The water protective membranes increase the reaction time because a wick member must be used to transport water into the membrane shell, thereby increasing the time required to dispose water inside the shell due to the relatively small cross-sectional area of the wick penetrating the shell. Further, although the membranes are semi-permeable to chlorine dioxide gas, the flow of chlorine dioxide gas is restricted through the membranes during gas generation thereby restricting “breathability” of the shell.

Another problem with prior art chlorine dioxide gas generators is that only one cavity is provided to receive a mixture of sodium chlorite and acid. The mixing of the reactants results in inconsistencies and varying contact ratios between the sodium chlorite and acid resulting in varying quantities of chlorine dioxide gas being generated when water engages the reactants.

The mixed internal components form different surface areas of sodium chlorite that engage acid relative to the wick member. When water initially engages the internal components adjacent to the wick member, then travels to internal components further from the wick member, varying amounts of sodium chlorite react with varying amounts of acid, thereby providing slower and/or incomplete reactions between the sodium chlorite and acid, resulting in wasted residual portions of each internal component which must be discarded, and which did not generate any chlorine dioxide.

U.S. Pat. No. 5,126,070, issued to Leifheit et al. on Jun. 30, 1992, discloses a rupturable or frangible pouch and an absorbent carrier for reacting a chlorite and an acid to form chlorine dioxide gas. The speed of chlorine dioxide gas formation is dependent upon the manual force applied to the package to combine the internal components.

U.S. Pat. No. 6,764,661, issued to Girard on Jul. 20, 2004, U.S. Pat. No. 10,105,461 issued to Schroeter, T., one of the current inventors of this application, discloses wick means extending into and connected to a membrane shell defining a compartment. A wick member extends outside of the compartment. The wick member absorbs water outside of the compartment and transports the water into the compartment to expose the components therein to water to produce chlorine dioxide gas.

In general, the prior art devices and methods do not provide sufficient surface area to fully utilize all of the supplied chemical and to cause a complete reaction between sodium chlorite and acid such that there are no “unused” portions of either component, which results in a less than maximum formation of chlorine dioxide gas. More specifically, the prior art devices resort to manual force or added components (wick means) to urge the engagement of sodium chlorite, acid and water instead of using the relatively large surface area of the packet containing the components to ultimately expose the components to an aqueous solution. Further, the prior art devices do not use a material for constructing the packets or shells that are capable of allowing a relatively large quantity of water to flow relatively quickly through the shell to engage the internal components, and that allows generated chlorine dioxide gas to escape relatively fast through the shell and into the surrounding air and/or water. Also, although the material of construction should allow water through the shell, the material must resist atmospheric moisture to prevent premature activation of the internal components. Another problem with the prior art is that the packets are not rigid and therefore change shape after disposing dry reactants into chambers, resulting in less than full chambers, non-uniform distribution of the dry chemicals in the packet, and dry chemicals that vary in configuration when the orientation of the packet is changed, thereby reducing chlorine dioxide generation, and allowing residual unused chemicals.

U.S. Pat. No. 10,105,461 issued to Schroeter, T., one of the current inventors of this application, discloses the current prior art kit for facilitating a chemical reaction. U.S. Pat. No. 10,105,461 is incorporated by reference in its entirety herein for all its teachings.

The above prior art of U.S. Pat. No. 10,105,461 has many deficiencies. Field use has demonstrated that the dissolvable member does not dissolve in a consistent manner within the device or across multiple devices. This leads to a reduced chlorine generation rate and subsequently lower maximum chlorine dioxide concentration in the space to be disinfected and a reduced amount of chlorine dioxide produced which wastes the unused raw materials.

A need therefore exists for an improved device for facilitating a chemical reaction which has a higher generation rate and higher maximum chlorine dioxide concentration without wasting as much unreacted raw materials.

### SUMMARY OF THE INVENTION

It is an object of the present invention to overcome many one of the disadvantages associated with prior art devices for facilitating a chemical reaction.

This specification discloses an improved packet of a kit for facilitating a chemical reaction comprising. The prior art features of a first packet member defining a first chamber portion with the first packet member being formed of a water permeable, first compressed cellulose material having a first average pore diameter.

The prior art packets have a second packet member defining a second chamber portion, said second packet member being formed of a water permeable, second compressed cellulose material having a second average pore diameter.

The specification further discloses that the prior art packet being improved upon has a first dry ingredient disposed in said first chamber portion and a second dry ingredient disposed in said second chamber portion.

It is further disclosed that said first packet member is attached to said second packet member with a dissolvable member disposed between and ultimately enclosed by said first packet member and second packet member; wherein said first dry ingredient and said second dry ingredient are configured to form chlorine dioxide in the presence of water.

The specification discloses that the improvement is the presence of a plurality of apertures passing through the dissolvable member.

It is further disclosed that the dissolvable member is fabricated from polyvinyl alcohol and capable of engaging the first ingredient and the second ingredient without a reaction.

It is also disclosed that the second cellulose material of the second packet member may be uncompressed and that the first cellulose material of the first packet member may be uncompressed.

It is further disclosed that the plurality of apertures is selected from the group consisting of slits, holes, cut-outs, pinholes, laser cut apertures, and combinations thereof. It is also disclosed that the apertures could be in the shape of a flap.

It is also disclosed that the dissolvable member with the plurality of apertures can be the same weight as a dissolvable member control without the plurality of apertures.

It is disclosed that the first chamber portion and the second chamber portion are dimensioned so as to accommodate a substantially equal amount in terms of bulk volume of said first dry ingredient and said second dry ingredient.

It is further disclosed that the first dry ingredient comprises dry sodium chlorite and the second dry ingredient comprises at least one dry acid selected from the group consisting of citric acid, boric acid, lactic acid, tartaric acid, maleic acid, malic acid, glutaric acid, adipic acid, acetic acid, formic acid, sulfamic acid, sulfuric acid, hydrochloric acid, phosphoric acid, phosphoric anhydride, sulfuric anhydride, maleic anhydride, calcium chloride, magnesium chloride, magnesium nitrate, lithium chloride, magnesium sulfate, aluminum sulfate, aluminum hydroxide, sodium acid sulfate, sodium dihydrogen phosphate, potassium acid sulfate, potassium dihydrogen phosphate, and sodium persulfate.

A kit for using the improved packet is also disclosed which includes a container (holder) for receiving the packet, said container being sized to receive a pre-determined amount of a liquid catalyst for facilitating a reaction between said first dry ingredient and said second dry ingredient within said packet.

### BRIEF DESCRIPTION OF THE DRAWINGS

These and other objects, advantages, and novel features of the present invention, as well as details of an illustrative embodiment thereof, will be more fully understood from the following detailed description and attached drawings, wherein FIGS. 1 to 12 and the nomenclature thereof are the same ones used in U.S. Pat. No. 10,105,461, with the figures of U.S. Pat. No. 10,105,461 identified as prior art:

FIG. 1 is a front elevation view of a prior art kit for facilitating a chemical reaction in accordance with the present invention. The kit includes a single packet in a holder.

FIG. 2 is a top view of the prior art single packet of FIG. 1.

FIG. 3 is a front exploded view of the prior art single packet of FIG. 2.

FIG. 4 is a sectional view taken along line 4-4 of FIG. 2.

FIG. 5 is a front sectional view of the prior art single packet of FIG. 3.

FIG. 6 is a front elevation view of an alternative prior art kit for facilitating a chemical reaction in accordance with the present invention. The packet component includes three packets.

FIG. 7 is a sectional view FIG. 6.

FIG. 8 is a top view of the prior art three packets of FIG. 6.

FIG. 9 is an exploded sectional view of the prior art three packets of FIG. 7.

FIG. 10 is an internal view of a prior art nested chamber packet in accordance with the present invention.

FIG. 11 is an internal view of a prior art multilayer packet in accordance with the present invention.

FIG. 12 is the prior art single packet sectional view of FIG. 4 but with an alternative configuration for the dissolvable member in accordance with the present invention.

FIG. 13 depicts typical shapes of apertures passing through a dissolvable member.

FIG. 14 is a graph showing the cumulative exposure over time of the working examples.

FIG. 15A depict the measured chlorine dioxide level at the moment in time of Working Example 1.

FIG. 15B depicts the measured chlorine dioxide level at the moment in time of Working Example 2

FIG. 16 depicts the cumulative chlorine dioxide level versus the time of measurement of the Control prior art package.

FIG. 17 depicts the chlorine dioxide level versus the time of measurement of the Control prior art package.

### DESCRIPTION OF THE PREFERRED EMBODIMENT

The elements of the figures are defined as:

Element 10 is a kit comprising holder 22 and packet 11 in which the chemical reaction occurs.

Element 11 points to the packet.

Element 12 points to an upper member of the packet.

Element 14 points to a lower member of the packet.

Element 15 points to stitching in the packet.

Elements 16 and 16a point to a dissolvable member of the packet.

Element 18 points to an upper chamber in the packet.

Element 20 points to a lower chamber in the packet.

Element 22 points to a holder of the kit.

Element 24 is an arcuate end portion.

Element 26 is a arcuate end portion opposite Element 24.

Element 28 is a central portion of the packet.

Element 30 is a first reactant, typically sodium chlorite.

Element 32 is the second reactant, typically an acid or acid mixture.

Element 34 is a recess in the holder.

Element 36 is an outer perimeter or periphery of the joined upper and lower members.

Element 38 is the upper chamber side of the dissolvable member.

Element 40 is the lower chamber side of the dissolvable member.

Element 42 is a multi-chamber (i.e. greater than two chambers) embodiment of the packet.

Element 44 points to the three upper chambers of the multi-chamber packet.

Element 46 points to the three lower chambers of the multi-chamber packet.

Element 47 is the stitching of the multi-chamber packet.

Element 60 is a nested chamber embodiment of the packet.

Element 62 is the inner chamber.

Element 64 is the middle chamber.

Element 66 is the outer chamber.

Element 68 is compressed cellulose.

Element 70 is compressed cellulose.

Element 72 is a compressed cellulose cloth.

Element 80 is the embodiment of a multi-layer onion packet.

Element 81 is the center core chamber.

Element 82 is the second reactant, typically an acid or acid mixture.

Element 83 is a compressed cellulose sponge.

Element 84 is a compressed cellulose cloth.

Element 90 is an alternative configuration of the dissolvable member.

Element 91 points to a dissolvable member.

Element 92 is a recess.

Element 94 is a conical wall.

Element 161 depicts a plurality of apertures which are slits in a straight line.

Element 162 depicts a plurality of apertures which are a combination of slits and pin holes along a straight line.

Element 163 depicts a plurality apertures of pin holes in a tight line.

Element 164 depicts a plurality of slits in a straight line. In this case the slits have a smaller aspect ratio and are closer together than the slits depicted by 161.

Element 165 depicts a plurality of round holes with the center removed.

Element 166 depicts a plurality of rectangle cutouts with the dissolvable material inside the cutout removed from the dissolvable member aligned in a straight line.

Element 167 depicts a plurality of flaps.

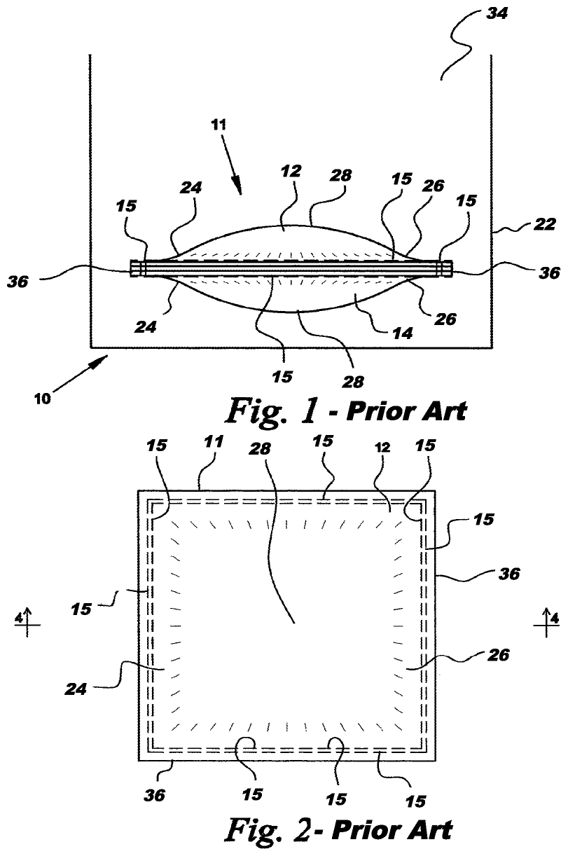

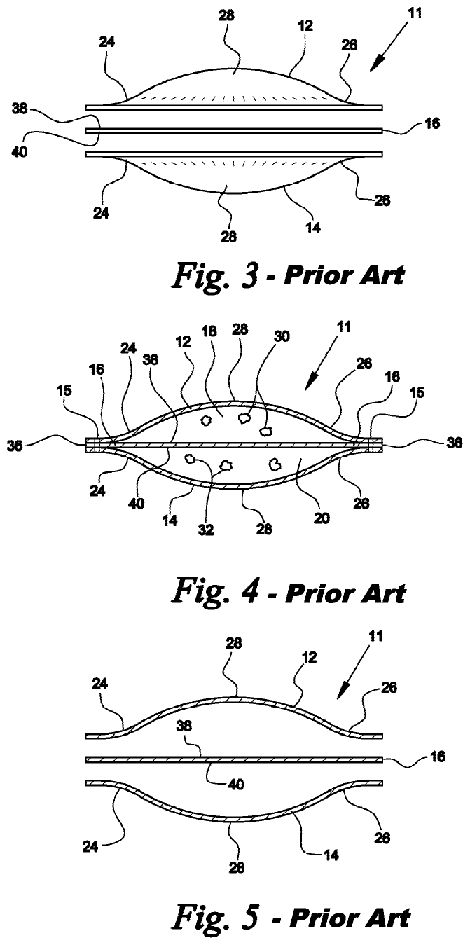

U.S. Pat. No. 10,105,461 refers to its FIGS. 1-5 as a kit containing a packet fabricated from compressed cellulose for facilitating a chemical reaction between a liquid catalyst and one or more dry reactants within the packet to produce a gas or liquid that is ultimately released into air or liquid released to its surroundings is denoted as numeral 10. It is important to recognize that some reactions produce a liquid, some reactions produce a gas which only exists in the gas phase, or a vapor which has a gas phase in equilibrium with a liquid phase.

One use for the kit 10 of the present invention is the generation of a gas by acid activation. Examples of acid activation include but are not limited to acid activation of a carbonate via calcium carbonate combined with citric acid in the presence of moisture to form carbon dioxide, acid activation of a sulfite via sodium bisulfite or potassium bisulfite with fumaric acid and/or potassium bitartrate in the presence of moisture to form sulfur dioxide gas, and acid activation of a nitrite via sodium nitrite or potassium nitrite in the presence of moisture to form nitrogen dioxide gas. The product of the reaction of the

According to U.S. Pat. No. 10,105,461, a preferred use of the kit 10 is the generation of chlorine dioxide gas for release into air or water. The kit 10 includes a single packet 11 fabricated from a compressed cellulose material. The compressed cellulose material causes the packet 11 to be rigid thereby preventing the packet from deforming or otherwise changing configuration after disposing dry reactants into chambers, resulting in continuously full chambers of dry chemicals that maintain a constant configuration within the packet 11, which causes consistent chlorine dioxide generation irrespective of packet 11 orientation. The single packet 11 includes an upper member 12 having a predetermined configuration, a lower member 14 having a predetermined configuration, a dissolvable member 16 disposed between and ultimately enclosed by the upper and lower members 12 and 14 such that upper and lower chambers 18 and 20 are formed having substantially equal volumes. The upper chamber 18 is substantially filled with sodium chlorite 30 and the lower chamber 20 is substantially filled with an acid or acid mixture 32. The device 10 further includes a holder member 22 for receiving a predetermined quantity of liquid catalyst such as water, and for receiving the joined upper and lower members 12 and 14 with the dissolvable member 16, sodium chlorite 30 and acid or acid mixture 32 therein.

U.S. Pat. No. 10,105,461 refers to FIG. 4 as disclosing the upper and lower members 12 and 14 including configurations having first and second arcuate end portions 24 and 26 with substantially planar central portions 28 therebetween. The arcuate end portions 24 and 26 cooperate with the central portions 28 to configure the upper and lower chambers 18 and 20 such that relatively large central portion volumes and relatively small edge portion volumes are formed in each chamber 18 and 20. The upper chamber 18 is substantially filled with a dry anhydrous sodium chlorite. The lower chamber 20 is substantially filled with a dry anhydrous acid 32, preferably citric acid; whereupon, the upper and lower members 12 and 14 are joined via stitching 15 (preferably a double stitch) or similar securing means, thereby sealing the upper and lower chambers 18 and 20 and enabling the joined upper and lower members 12 and 14 to be disposed in a predetermined volume of water in the holder member 22 such that the lower member 14 engages the water first. The lower member 14 quickly absorbs a volume of water substantially greater than the upper member 12. The upper and lower members 12 and 14 are sized and configured to cooperate and swell to absorb all the predetermined volume of water disposed in the holder member 22.

Referring to FIG. 1, U.S. Pat. No. 10,105,461 discloses that the configuration of the holder member 22, when taking a top view of the member 22, corresponds to the configurations of the joined upper and lower members 12 and 14 such that a relatively rectangular configuration is presented by both the joined members 12 and 14, and the holder member 22. The configurations of the upper and lower members 12 and 14 promote a rate of absorbing water via the lower member 14 and the acid or acid mixture 32 that ultimately results in an acid slurry in the lower chamber 20; and a rate of absorbing water via a periphery 36 of the upper member 12 joined to the periphery of the lower member 14, then into the sodium chlorite mixture to ultimately form a slurry in the upper chamber 18. The sodium chlorite slurry forms in the upper chamber 18 at a slower rate than the formation of the acid slurry in the lower chamber 20. Both slurries ultimately cooperate to dissolve the dissolvable member 16. The holder member 22 includes a recess 34 having a substantially rectangular configuration. The recess 34 has longitudinal and lateral dimensions slightly larger than corresponding longitudinal and lateral dimensions forming an outer perimeter or periphery 36 of the joined upper and lower members 12 and 14.

According to U.S. Pat. No. 10,105,461 the holder member 22 receives a predetermined quantity of water and said joined upper and lower members 12 and 14 with said dissolvable member 16 therebetween. Said lower and upper member 14 and 12 configurations cooperate to allow water to engage the dry acid or acid mixture 32 in the lower chamber 20 followed by the now acidic liquid catalyst in the lower member 14 being absorbed by the upper member 12 through periphery contact at the sewn edges 36 and engaging the substantially dry sodium chlorite 30 in the upper chamber 18, thereby beginning the conversion of sodium chlorite 30 to chlorine dioxide and to ultimately form slurries that completely dissolve the dissolvable member 16, thereby allowing said slurries to engage in the continuous reaction of the acid slurry and sodium chlorite slurry to continuously produce chlorine dioxide gas until all chemicals have been exhausted. The chlorine dioxide gas passes through the upper and lower members 12 and 14 and into a space to be disinfected and/or deodorized. The upper and lower members 12 and 14 are dimensioned and configured to cooperate with selected quantities of dry sodium chlorite 30 and dry acid 32 mixtures to generate a predetermined quantity of chlorine dioxide gas over a predetermined time period. The predetermined quantity of water is absorbed relatively quickly by the lower member 14, then absorbed by the upper member 12 through the sewn edges 36 after the joined lower and upper members 14 and 12 are disposed in the water.

U.S. Pat. No. 10,105,461 teaches that the dissolvable member 16 allows the slurries to engage and generate chlorine dioxide gas that passes mainly through the upper member 12 with a relatively small amount of chlorine dioxide gas passing through the lower member 14. The chlorine dioxide gas exits the joined upper and lower members 12 and 14, then naturally flows into a space to be disinfected and/or deodorized. The upper and lower members are dimensioned and configured to cooperate with selected quantities of dry sodium chlorite and dry acid or acid mixtures to generate a predetermined quantity of chlorine dioxide gas over a predetermined time period. The predetermined quantity of water is absorbed relatively quickly by the lower and upper members 14 and 12 upon being disposed in a holder member recess 34 having dimensions slightly larger than corresponding dimensions of the periphery 36 of the joined upper and lower members 12 and 14. The configuration of the upper and lower members 12 and 14, allow a bottom compressed sponge cloth to engage the water and expand and be reconfigured such that the edges are contorted upward creating a cupping action or concave up configuration, resulting in a substantially wet acid engaging one side of the dissolvable member 16 and a substantially dry sodium chlorite engaging the opposite side of the dissolvable member 16. The now expanded bottom sponge cloth cooperates with the upper compressed sponge such that when the upper compressed sponge absorbs sufficient now acidified water to fully expand, the bottom sponge cloth reverts to a planar configuration to dispose the reactants of the upper and lower chambers 18 and 20 closer together. The upper and lower members 12 and 14 cooperate to allow a predetermined quantity of liquid catalyst to penetrate the lower member 14 and engage the dry acid reactant in the lower chamber 20.

The packet 11 of U.S. Pat. No. 10,105,461 is ultimately disposed in the liquid catalyst such that the lower member 14 or bottom compressed sponge cloth engages the liquid catalyst or water first, and expand and be reconfigured such that the edges 36 are contorted upward creating a cupping action or concave up configuration, resulting in a substantially wet acid 32 engaging one side of the dissolvable member 16 and a substantially dry sodium chlorite 30 engaging the opposite side of the dissolvable member 16. The now expanded bottom cellular cloth 14 (or sponge cloth) cooperates with the upper compressed sponge 12 such that when the upper compressed sponge 12 absorbs sufficient now acidified water to fully expand, the bottom sponge cloth 14 reverts to a planar configuration to dispose the reactants 30 and 32 of the upper and lower chambers 18 and 20 closer together. The lower and upper members 14 and 12 cooperate to allow a predetermined quantity of liquid catalyst to penetrate the lower member 14 and engage the acid reactant 32 in the lower chamber 20 followed by the now acidic liquid catalyst in the lower chamber 20 being absorbed by the upper member 12 through periphery contact at the sewn edges 47, the acidic liquid catalyst then engaging the substantially dry reactant 30 in the upper chamber 18, thereby beginning the conversion of sodium chlorite 30 to chlorine dioxide and ultimately forming slurries that completely dissolve the dissolvable member 16 to allow the slurries to engage in the continuous reaction of the chlorine dioxide until all chemicals have been exhausted.

The dissolvable member 16 of U.S. Pat. No. 10,105,461 preferably has longitudinal and lateral dimensions relatively smaller than corresponding longitudinal and lateral dimensions of the upper and lower members 12 and 14, thereby allowing the dissolvable member 16 to be totally encased between the upper and lower members 12 and 14 after the members 12 and 14 are joined via water resistant thread sewn about the periphery 36 of cooperating edge portions of the upper and lower members 12 and 14, or similar joining means well known to those of ordinary skill in the art. A myriad of materials may be used to fabricate the dissolvable member 16 including, but not limited to starch, gelatin and the preferred material of fabrication is film of a polyvinyl alcohol/starch that are capable of withstanding the dry chemical mixtures until activation by the liquid catalyst. A non-absorbent fiberglass cloth, mesh or weave, or similar non-absorbent, non-soluble weave may be included in the dissolvable member 16 to strengthen the dissolvable member 16 material and/or to slow down or otherwise control the rate of reaction between upper and lower chambers 18 and 20, thereby controlling the amount of water that mixes with the sodium chlorite 30 and the acid or acid mixture 32.

The upper member 12 of U.S. Pat. No. 10,105,461 is fabricated from a biodegradable, compressed cellulose sponge material having multiple pores that are closed when dry and open when wet. Preferably the upper member 12 material is manufactured by 3M Company (Minneapolis, MN, USA) and Spontex Company (Columbia, TN, USA), both well known to those of ordinary skill in the art. The lower member 14 is fabricated from a biodegradable, compressed cellulose cloth material having multiple pores substantially smaller in size than the pores of the cellulose sponge material of the upper member 12 pores. The lower member 14 material is manufactured from 3M and Spontex Companies. The upper and lower member 12 and 14 pores are closed when dry and open when wet. The closed pores of the upper and lower members 12 and 14 prevent the sodium chlorite and acid or acid mixture 30 and 32 from combining with moisture to start a premature reaction and/or from escaping the packet before activation. When the closed pores of the upper and lower members 12 and 14 open, the generation of chlorine dioxide gas is initiated and allowed to escape to through the upper and lower members 12, thereby preventing a pressure buildup of the generated gas, which can result in the spontaneous combustion or explosion of the chlorine dioxide gas.

In some applications a cellulose cloth which is not compressed is used instead of the compressed cloth. This is referred to as an uncompressed cloth.

The higher density of pores of the lower member 14 of U.S. Pat. No. 10,105,461 allow the lower member 14 to absorb and hold more water than the pores of the upper member 12. The upper member 12 pores become relatively larger than the lower member 14 pores when wet, thereby allowing a relatively large quantity of chlorine dioxide gas to escape from the upper member 12 in comparison to the lower member 14. The primary purpose for the pores of the upper member 12 is for gas release, and a secondary purpose for the pores being the absorbing of water. The primary purpose for the pores of the lower member 14 is for water absorbing, and a secondary purpose for the pores being gas release. The lower member 14 not only absorbs water via the pores, but also via the fiber material that forms the lower member 14. The sponge material of the upper member 12 has less fiber than the lower member 14 and correspondingly absorbs less water. Besides the smaller pores of the lower member 14 impeding chlorine dioxide gas flow, engagement between the lower member 14 and the holding member 22 also restricts chlorine dioxide gas flow. The upper and lower members 12 and 14 hold the absorbed water during the entire reaction time for forming chlorine dioxide gas. The surface areas for the upper and lower members 12 and 14 are relatively small before submersion and relatively large when exposed to water during the entire reaction time for forming chlorine dioxide gas.

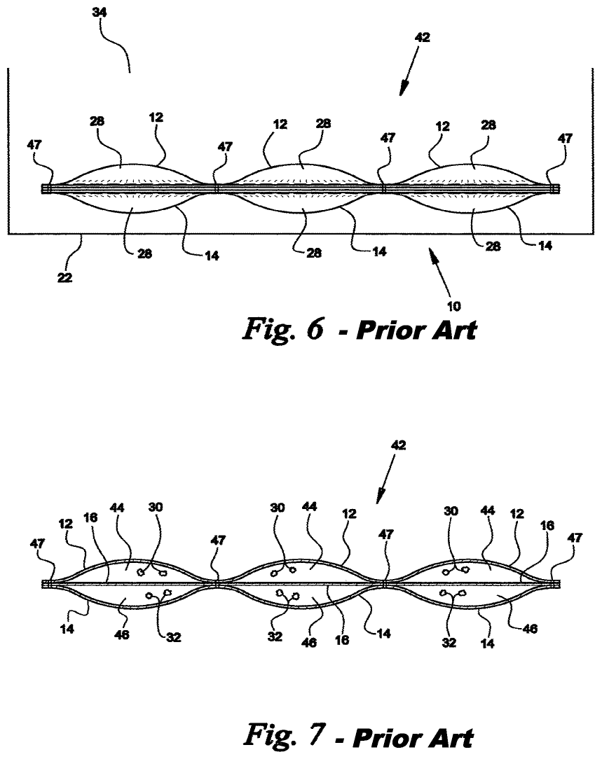

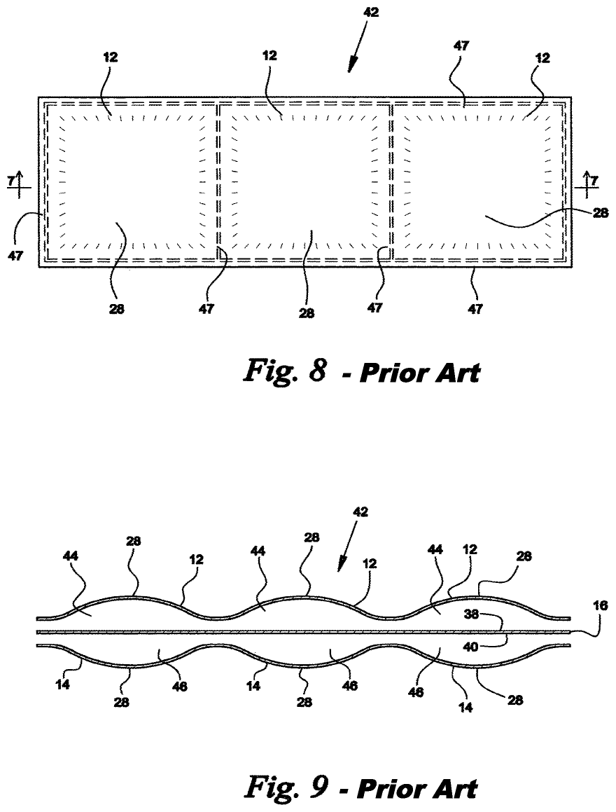

Referring to FIGS. 6-9, U.S. Pat. No. 10,105,461 discloses a multi-chamber packet 42, which is used for releasing chlorine dioxide gas into air, is depicted with three upper chambers 44 and three lower chambers 46. Each chamber 44 and 46 is substantially the same configuration and dimensions as the corresponding chambers 18 and 20 of the single packet 11 of FIGS. 1-5. Each chamber 44 and 46 has a peripheral stitching 47 (preferably a double stitch) that captures the sodium chlorite or acid or acid mixtures in respective sealed and separated chambers 44 and 46.

The multi-chamber packet 42 of U.S. Pat. No. 10,105,461 provides for more generation of chlorine dioxide gas from the multi-chamber packet 42 compared to the single packet 11, when each individual chamber of the multi-chamber packet 42 is substantially equal in volume to the single packet 11. Obviously, a relatively larger single packet 11 could be used to generate more chlorine dioxide gas; however, a larger single packet 11 is not efficient due to the corresponding larger quantity of sodium chlorite 30 in the upper chamber 18 ultimately combining with water to form a “caked” or hardened central core surrounded by relatively wet powder. The hardened core of sodium chlorite 30 prevents the acid or acid mixture 32 from fully dissolvable and activating the sodium chlorite 30 after the acid or acid mixture 32 dissolves the dissolvable member 16 and engages the sodium chlorite 30, resulting in wasted quantities of both the sodium chlorite 30 and the acid or acid mixture 32. The separated chambers 44 and 46 of the multi-chamber packet 42 provide smaller chamber quantities of the sodium chlorite 30 and acid or acid mixture 32 for promoting faster and more complete reactions, thereby generating more chlorine dioxide gas from the pre-selected quantity of all sodium chlorite 30 and acid or acid mixture 32 in all the chambers 44 and 46 of the multi-chamber packet 42, than the amount of chlorine dioxide gas generated from the same pre-selected quantity of sodium chlorite 30 and acid or acid mixture 32 disposed in larger single chambers 18 and 20 in a correspondingly larger single packet 11.

The single packet 11 of U.S. Pat. No. 10,105,461 in FIGS. 1-5 and the multi-chamber packet 42 of FIGS. 6-9, may be used to release chlorine dioxide gas into water by using a higher density cellulose material with greater numbers and greater density of smaller pores for the upper members 12 forming the upper chambers 18 and 44. The compressed cellulose material for the upper member 12 is substantially the same as the cellulose material (manufactured from 3M and Spontex Companies) used for the lower members 14 forming the lower chambers 20 and 46. The higher pore density of the compressed cellulose cloth of the upper and lower members 12 and 14 allows water to pass therethrough to form a sodium chlorite slurry in the upper chambers 18 and 44 and an acid slurry in the lower chamber 20 and 46, whereupon, the slurries dissolve the dissolvable members 16 and ultimately mix and react to release chlorine dioxide gas through the pores of the cellulose material before the slurries diffuse or otherwise “escape” from the upper chambers 18 and 44 and the lower chambers 20 and 46, and into the surrounding liquid mass or water.

The compressed cellulose cloth of the upper and lower members 12 and 14 of U.S. Pat. No. 10,105,461 includes an outer surface or “skin” for retaining water in the pores of the cloth. The skin replaces the open pores on the surface of the cloth. More specifically, there are no open pores on the surface of the cloth, but there are ultimately small open pores inside the cell structure of the inner layers of the cloth material, thereby allowing generated chlorine dioxide gas to escape from the packets 11 and 42 via the open pores and through spaces between the fibers of the caused by water contacting the cloth material. Both the single packet 11 and the multi-chamber packet 42 require a weight secured thereto to maintain the respective packet under water in a vertical or horizontal orientation. Attaching the weight to the respective packet is well known to those of ordinary skill in the art.

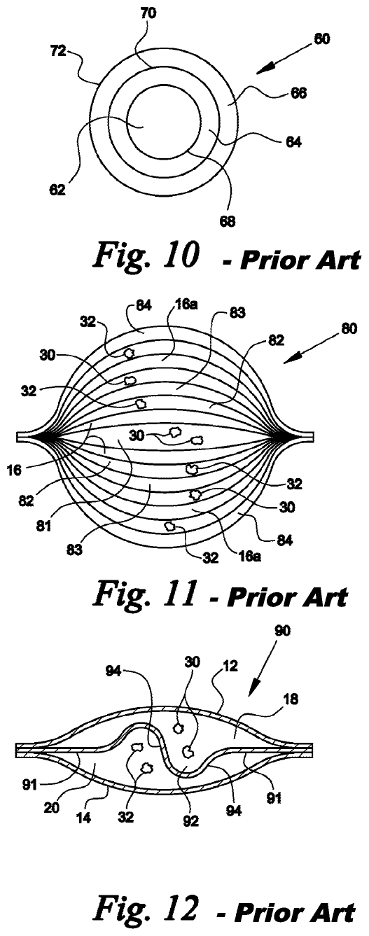

Referring to FIG. 10, U.S. Pat. No. 10,105,461 discloses a nested chamber packet 60 is depicted for use when chlorine dioxide is released in water. The nested chamber packet 60 must be maintained under water via a weight or similar means as detailed above for the multi-chamber packet 42. FIG. 10 includes three nested chambers, an inner chamber 62, a middle chamber 64 and an outer chamber 66. The inner chamber 62 includes sodium chlorite 30 surrounded by a compressed cellulose sponge 68. The middle chamber 64 includes sodium chlorite 30 surrounded by a compressed cellulose sponge 70. The outer chamber 66 includes an acid or acid mixture 32 surrounded by a compressed cellulose cloth 72. The cellulose cloth 72 slowly allows water to enter the outer chamber 66 and form an acid slurry that ultimately penetrates the sponge 70 of the middle chamber 64 followed by the acid slurry penetrating the sponge 68 of the inner chamber, thereby extending the release time for the chlorine dioxide gas from the nested chamber packet 60 to sanitize or disinfect a water volume, pools and cooling towers for example, for a time period much longer than the aforementioned single and multi-chamber packets 11 and 42.

Referring to FIG. 11, U.S. Pat. No. 10,105,461 a multi-layer “onion” packet 80 is depicted for increasing the release time for chlorine dioxide into water. The multi-layer packet 80 is maintained under water via a weight or similar means as detailed above. The center core chamber 81 contains sodium chlorite 30 and is defined by two dissolvable members 16. The next layer 82 is an acid or acid mixture 32 captured between the two dissolvable members 16 and two compressed cellulose sponge members 83. The next layer is sodium chlorite 30 captured between the two cellulose sponge member 83 and two dissolvable members 16a. The next layer is an acid or acid mixture 32 captured between the two dissolvable members 16a and two cooperating compressed cellulose cloth members 84 that form an outer shell.

According to U.S. Pat. No. 10,105,461, irrespective of the type of packet used, all packets should be placed in a moisture resistant package to prevent the premature combination and reaction of the sodium chlorite and acid or acid mixtures. For safety, the holder member should include a cover to prevent water containing chlorine dioxide gas from escaping and/or improperly disposed, and for maintaining chlorine dioxide as inside the holder member 22.

The aforementioned packets of U.S. Pat. No. 10,105,461 can have a myriad of sizes and configuration for a predetermined volume of air or water to be disinfected and deodorized. However, the chamber sizes and the corresponding ratios for the respective chemical mixtures within the chambers will remain substantially constant. For example, an upper chamber 18 sized to contain a dry sodium chlorite mixture of five grams will be joined to a lower chamber 20 having a dry acid or acid mixture quantity of substantially about 16.5 grams of citric acid anhydrous. The quantity of water disposed in the holder member 22 to react with the above quantities is substantially about sixty milliliters. The dimensions of the compressed cellulose sponge forming the upper member 12 is substantially about 2⅝×3¾× 5/16 inches. The dimensions of the compressed cellulose cloth forming the lower member 14 is substantially about 2⅝×3¾× 5/16 inches. The dimensions of the dissolvable member 16 is relatively smaller than substantially about 2⅝×3¾× 1/32 inches.

The method disclosed in U.S. Pat. No. 10,105,461 for fabricating the single packet 11 includes the following steps:

Disposing said polyvinyl alcohol material upon said compressed cellulose cloth;

Disposing said compressed cellulose sponge upon said polyvinyl alcohol material;

Securing together engaging peripheral portions of said compressed cellulose sponge, said compressed cellulose cloth and said polyvinyl alcohol such that a side portion remains open;

Providing substantially about sixteen and one-half grams of citric acid in a room having a humidity level at or less than twenty percent;

Disposing half of said first mixture between said compressed cellulose cloth and said polyvinyl alcohol material;

Disposing a second mixture consisting of five grams of sodium chlorite between said compressed cellulose sponge and said polyvinyl alcohol material;

Disposing the remaining half of said first mixture between said compressed cellulose cloth and said polyvinyl alcohol material;

Sealing said open side portion such that said first and second mixtures are isolated and sealed between respective walls formed from said compressed cellulose sponge, said compressed cellulose cloth and said polyvinyl alcohol, thereby forming a chlorine dioxide generating device;

Activating said chlorine dioxide generating device via sixty milliliters of relatively warm water disposed in a container, said chlorine dioxide generating device being disposed in said container such that said compressed cellulose cloth forms a lower portion of the device that engages the water before said compressed cellulose sponge engages the water, thereby causing chlorine dioxide gas to be emitted from said device until all reactions have exhausted and said water has been completely absorbed by said compressed cellulose.

U.S. Pat. No. 10,105,461 refers to its FIG. 12, a sectional side view of a single packet 11 which depicts an alternative configuration for the dissolvable member 16 of FIG. 4, the alternative configuration being denoted as numeral 90. The dissolvable member 91 can be used with the single packet 11 or the multi-chamber packet 42 for generating chlorine dioxide gas into air or water. The dissolvable member 91 includes an undulating or “wave” configuration that is formed via the above detailed steps for fabricating the single packet 11. The dissolvable member 91 provides a trough or recess 92 that receives sodium chlorite 30 therein. The upper and lower chambers 18 and 20 are completely filled with sodium chlorite 30 and acid or acid mixture 32, thereby forcibly maintaining sodium chlorite 30 in the recess 92 irrespective of the orientation of the packet 11 and 42. The conical wall 94 of the recess 92 of the dissolvable member 91 provides more surface area than a planar dissolvable member 16, thereby increasing cooperating quantities of sodium chlorite 30 and acid or acid mixture 32 disposed adjacently on opposite sides of the dissolvable member 91. When the dissolvable member 91 is dissolved by acid and sodium chlorite slurries, the increased quantities of sodium chlorite and acid slurries that immediately mix together ultimately generates chlorine dioxide gas at a faster rate than the gas rate generated by relatively smaller slurry quantities that mix after a planar dissolvable member 16 is dissolved. Thus, the gas generation rate for the packets 11 and 42 can be increased or decreased by correspondingly increasing or decreasing the surface area of the recess 92, and the surface area of the recess 92 is varied by correspondingly changing the configuration and/or dimensions of the dissolvable member 91.

It has surprisingly been discovered that if the dissolvable member has a plurality of apertures passing through it, the dissolvable member disintegrates much more uniformly and quickly than the same membrane without the apertures. The use of a dissolvable membrane with apertures through the membrane increases the rate at which chlorine dioxide is generated, achieves a higher maximum chlorine dioxide concentration, and presents a more efficient use of the raw materials.

In fact, it has also been discovered, as discussed in the experimental section, that the use of apertures can tailor the chlorine dioxide evolution for various purposes.

Significantly higher initial levels of chlorine dioxide in the space to be disinfected have been observed when the dissolvable member has apertures when compared to a device where the dissolvable member has no apertures

An aperture is any passage which begins on one side of the membrane and passes through the membrane to the opposite side. The aperture-area per unit area such as square inches or square centimeters or square millimeters is called the aperture-inch density. The aperture-area density is an optimizable variable and varies upon the size of the device, the amount of raw materials in the device and the porosity/water transmission rate of the upper and lower members (12 and 14). If the aperture is a cutout hole for example, the aperture area is the total area which has been removed per square unit of area. In the case of a slit, where no material is removed, the aperture area is expressed as aperture-inches or slit-inches.

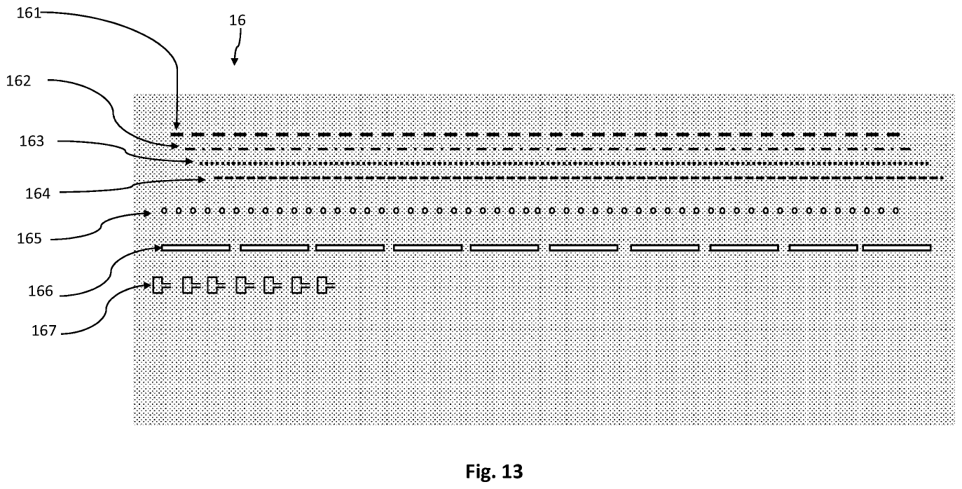

As shown in FIG. 13, the shape of the aperture is also a design choice to be optimized. In one embodiment the apertures are holes without a cutout portion, such as those made by a needle. In another embodiment, the apertures are holes but with a portion removed or cutout from the hole, i.e. a cutout formed by a device such as that used to punch holes in leather. In another embodiment, the apertures are slits having a specific slit length and slit width as measured on the device creating the slit. The slit length is always the longer dimension. A slit does not have any material removed. A slit has an aspect ratio greater than 1.1, while a hole has an aspect ratio of less than or equal to 1.1 and greater than or equal to 1.0.

161 of FIG. 13 depicts slits in a straight line. 162 of FIG. 13 depicts the combination of slits and pin holes along a straight line. 163 of FIG. 13 depicts pin holes in a tight line. 164 of FIG. 13 depicts slits in a straight line. In this case the slits have a smaller aspect ratio and are closer together than the slits depicted by 161. 165 of FIG. 13 depicts round holes with the center removed. These are called cutout holes. 166 of FIG. 13 depicts a rectangle cutout with the dissolvable material inside the hole removed from the dissolvable member aligned in a straight line. The flap (167 in FIG. 13) is another type of aperture. While shown as a slit in the shape rectangle in the figure it could just as easily be a semi-circle slit. A circle formed by a slit having an arc 330 Degrees of the circumference is an example of a flap. In this manner, the slit keeps the flap in place to keep the ingredients apart but rapidly dissolves creating a hole for more rapid movement across the membrane. The flap can be defined as a slit which is not a straight line.

The aperture opening can be varied as well. Preferably no material is removed from the dissolvable member when forming the aperture. In this manner the dissolvable member maintains its function as a physical separation barrier between the two chambers. One way to express that no material has been removed during the forming of the apertures is that the dissolvable member with the plurality of apertures is the same weight of a dissolvable member control without the plurality of apertures. The dissolvable member control is of the same material dimensions as the dissolvable member.

Should the aperture opening which has membrane material removed be desired, the aperture opening should be smaller than the average diameter of about 95% of the particles, or exactly 95% of the in either the upper or lower chamber. In this manner, some physical mixing may occur, but not enough to create a premature reaction.

As an optimizable variable, the aperture opening is preferably smaller than the average diameter of 50% of the particles in either the upper or lower chamber, with smaller than the average diameter of 60% of the particles in either the upper or lower chamber being more preferred, with smaller than the average diameter of 70% of the particles in either the upper or lower chamber being even more preferred, with smaller than the average diameter of 80% of the particles in either the upper or lower chamber being also more preferred, and with smaller than the average diameter of 90% of the particles in either the upper or lower chamber being again, even more preferred.

There will also be an aperture spacing for each aperture which is the distance between apertures along a line connecting at least three apertures. An aperture can have different spacings on different lines.

The slits can made by any suitable device such as a laser, a knife, a rolling wheel like a scoring wheel, scissors, needle(s), pin(s), and the like.

In one embodiment the first dry ingredient comprises dry sodium chlorite.

In one embodiment the said second dry ingredient comprises at least one dry acid selected from the group consisting of citric acid, boric acid, lactic acid, tartaric acid, maleic acid, malic acid, glutaric acid, adipic acid, acetic acid, formic acid, sulfamic acid, sulfuric acid, hydrochloric acid, phosphoric acid, phosphoric anhydride, sulfuric anhydride, maleic anhydride, calcium chloride, magnesium chloride, magnesium nitrate, lithium chloride, magnesium sulfate, aluminum sulfate, aluminum hydroxide, sodium acid sulfate, sodium dihydrogen phosphate, potassium acid sulfate, potassium dihydrogen phosphate, and sodium persulfate.

### Experimental

The ability of the aperture arrangement to modify the rate of reaction and the amount of material reacted was demonstrated by activating two working examples (WE-1 and WE-2) and measuring the chlorine dioxide levels relative to time during an 8-hr. cycle.

The working examples were the prior art packets with apertures in the dissolvable membrane

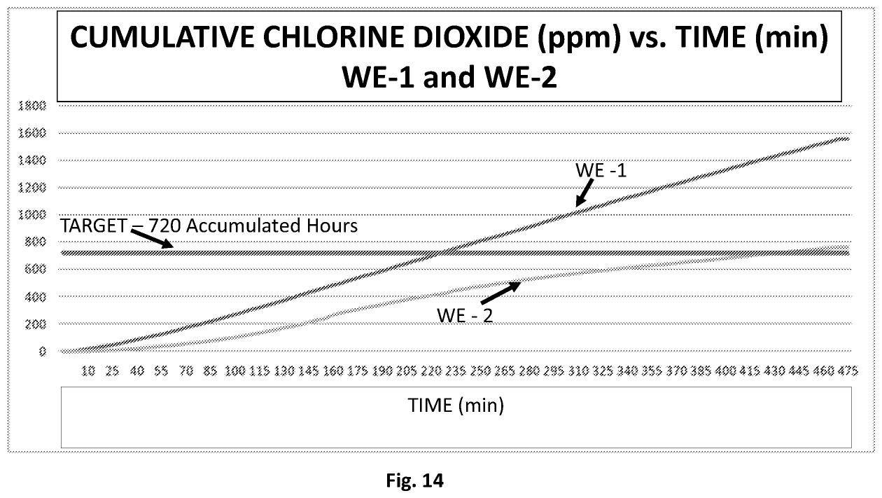

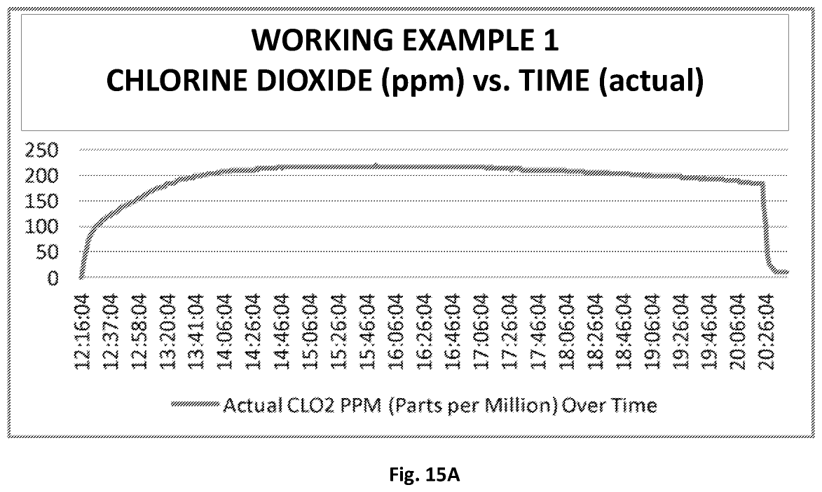

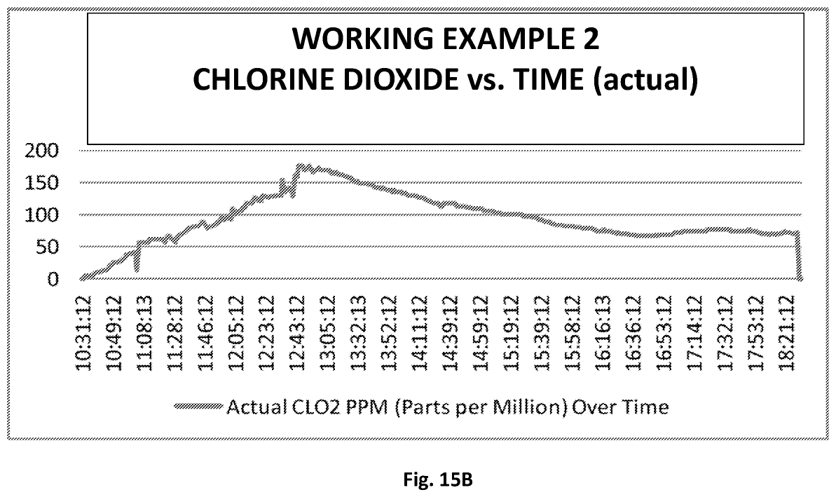

From this data the cumulative amount of chlorine dioxide generated during the 8-hr. cycle can be determined as shown in FIG. 14 and the chlorine dioxide concentration profile across the 8-hr. cycle can be determined (FIGS. 15A and 15B).

The testing protocol consisted of the following steps; 1) pre-conditioning of the closed space to reach a temperature and relative humidity level range between 10°-48.9° C. (50°-120° F.) and 65%-99% R.H. respectively; the diffusion phase of chlorine dioxide gas in order to reach the desired concentration level; a dwell period called exposure where the gas sits for a period of time in order to obtain the desired kill level; and finally, aeration or scrubbing to remove the gas safe levels.

The total exposure dosage, referred to as ppm-hours of accumulated exposure time, is the determining factor in sterilization, disinfection or sanitization cycle efficacy when using chlorine dioxide gas. Any concentration of gas can be used as long as it is held for the proper amount of time within specified atmospheric conditions in order to achieve the desired total ppm-hour exposure dosage.

The industry standard is 720 ppm-hours of accumulated exposure time which is the amount required to achieve a 6-log sporicidal reduction was established for this test using ClorDiSys performance standards. ClorDiSys is recognized as a worldwide leader in decontaminating critical aseptic environments using chlorine dioxide gas. Their performance metric of 720 ppm-hours of accumulated exposure time has been extensively peer reviewed and independently confirmed by many leading government and private research organizations. It has been demonstrated that a 6-log sporicidal reduction can be obtained with as low as 450 ppm-hours of accumulated exposure time.

The experiment utilized the following equipment:

Working Example 1: a Chlorine Dioxide Delivery System (kit) as described and claimed with the dissolvable member of 9 inches long and 3 inches wide with 4 rows of apertures (slits). The rows were spaced 0.75 inches apart running the length of the dissolvable member. Each slit was 0.5 inch long and 0.25 inches apart along the row. This equated to 1.5 slits per inch (0.75 slit-inches per inch). This equated to a density of 27 inches that were slit per 27 square inches of dissolvable member (a density of 1.0 slit-inches per square inch). 100 grams of a mixture of citric acid and sodium chlorite reactants were used.

Working Example 2: a Chlorine Dioxide Delivery System (kit) as described and claimed with the dissolvable member of 9 inches long and 3 inches wide with 4 rows of apertures (slits). The rows were spaced 0.75 inches apart running the length of the member. Each slit was 0.25 inch long and 0.25 inches apart along the row. This equated to approximately 2 slits per inch (0.5 slit-inches per inch). This equated to a density of 18 inches that were slit per 27 square inches of dissolvable member (a density of 0.67 slit-inches per square inch). 100 grams of the same reactant mixture as that of WE-1 were used.

The testing space was a containment chamber dimensioned 10 feet long×6 feet wide×7 feet having an internal volume of 321 Cu. Ft.

The gas concentration was measured using a calibrated ClorDiSys (Branchburg, NJ USA) EMS gas concentration monitoring system.

The ClorDiSys temperature and humidity monitoring system was used to measure the humidity and temperature of the chamber.

A ClorDiSys air scrubber was used for chemical neutralization after the 8 hr cycle.

Safety was ensured by one set of PPE safety equipment and a handheld PortaSens chlorine dioxide gas safety sensor available from ClorDiSys, Branchburg, NJ USA.

Humidity was controlled with a humidity generator with adjustment sensor.

Air was circulated with a low volume circular fan.

Crosstex (Hauppauge, New York, USA) biological indicators with prepared culture media containing Geobacillus stearothermophilus were used to detect the efficacy.

The temperature and humidity levels inside the test chamber were controlled to 68 to 72° and 72-85% RH.

Both working examples were activated using distilled water at a temperature of 67-69° F.

For each test, two biological indicators inoculated with Geobacillus stearothermophilus 10⁶ spores were placed inside the fumigation chamber. A 10⁶ or Log 6 efficacy level requires that the treatment provide a 99.9999% reduction of the bacterial spores. Geobacillus stearothermophilus spores are one of the most difficult spores to kill.

The internal chlorine dioxide concentration level was monitored throughout the process with a ClorDiSys environmental monitoring system (EMS) utilizing a precise UV-VIS spectrophotometer. Sample tubing was run to a remote area inside the chamber allowing a continuous sample to be pulled from the chamber so that the gas concentration level was monitored and logged at 1-minute intervals.

The chamber was decontaminated after 8 hrs. After completion of the decontamination process, all biological indicators were removed from the treatment chamber, inserted into the media, and incubated for 3 days per the manufacturer's testing requirements. The control indicator turned yellow, indicating biological growth while there was no change in color of the indicators corresponding to the working examples establishing that the required efficacy had been achieved.

As can be seen from FIG. 14, WE-1 with the greater slit-inches per inch exhibited a faster reaction and more thorough reaction of the reactants. By 3 hours the chamber with WE-1 was exposed to 750 ppm-hrs whereas it took almost the full 8 hrs for WE-2 to reach 750 ppm-hrs exposure. This reduces the treatment time by 50%. Alternatively, less reactants can be used, and less unreacted reactants to be disposed of.

The profiles of the concentration (FIGS. 15A and 15B) are interesting. It should be noted that these profiles were measured on gas drawn from an obscure part of the chamber, not near the device. The profile of WE-1 shows a very rapid generation and onset of the reaction. The implications are that the user should be mindful of the need to exit the area very quickly if there is no Personal Protective Equipment (PPE) in use. The profile of WE-2 shows a much slower reaction rate which allows the user more time to exit the area.

The improvement over the prior art used the same protocol as above except that the dissolvable membrane of the control had no apertures.

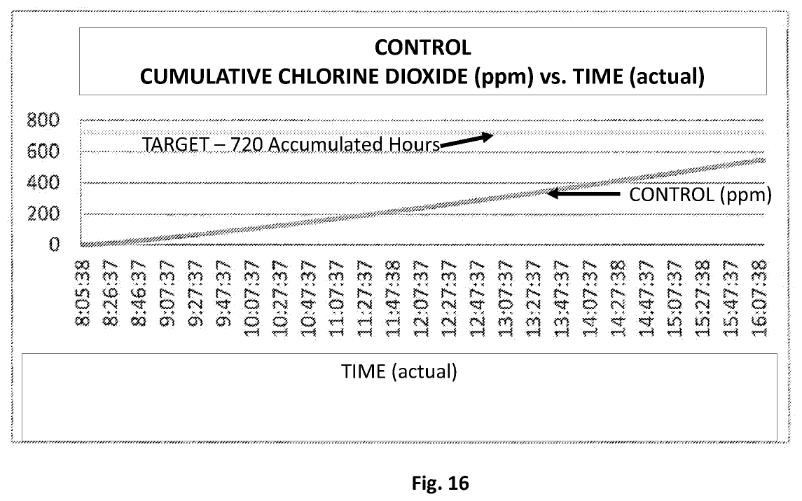

As can be seen from FIG. 16, the cumulative amount of chlorine dioxide did not reach the target of 720 accumulated hours in the 8 hr. period of the test. Contrast that with the Working Examples which did reach the target with 8 hrs.

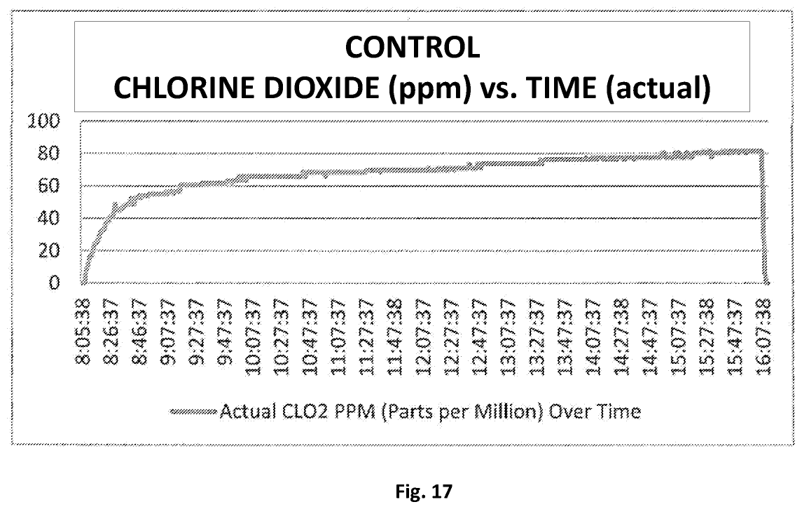

The improvement on the reactivity is demonstrated in FIG. 17 showing that maximum amount of chlorine dioxide at any given point in time was no greater than 80 ppm which be contrasted with WE-1 and WE-2 with 220 and 175 ppm, respectively.

TABLE 1 below summarizes the results over the 8 hr. test period as approximated from the Figures. As shown, the more slit-inches (or the more apertured the dissolvable member), the more ingredients are reacted in a given period of time.

TABLE I
SUMMARY OF RESULTS

| | Aperture | | | |
|---|---|---|---|---|
| | Density | Maximum | Time to 720 ppm | Accumulated |
| Identifi- | (Slit Inches | ClO₂ | accumulated | ClO₂ (ppm) |
| cation | per Sq. Inch) | (ppm) | ClO₂ (ppm) | in 8 hrs. |
| Control | None | 80 | Not Reached | 550 |
| WE-2 | 0.67 | 175 | 7.3 | 780 |
| WE-1 | 1.0 | 220 | 3.8 | 1550 |

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims. It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled.

1-18. (canceled)

19. A device for facilitating a chemical reaction comprising:
a first packet member defining a first chamber portion,
said first packet member being formed of a first water permeable, cellulose material;
a second packet member defining a second chamber portion, said second packet member being formed of a second water permeable, cellulose material;
a first dry ingredient disposed in said first chamber portion;
and a second dry ingredient disposed in said second chamber portion;
said first packet member being attached to said second packet member with a dissolvable member disposed between and ultimately enclosed by said first packet member and second packet member; wherein said first dry ingredient and said second dry ingredient are configured to form a gas, a vapor or liquid, or combination thereof in the presence of water;
and wherein there are a plurality of apertures passing through the dissolvable member.

20. The device of claim 19, wherein the plurality of apertures is selected from the group consisting of slits, holes, cut-outs, pinholes, laser cut apertures, flaps and combinations thereof.

21. The device of claim 19, wherein the dissolvable member with the plurality of apertures is the same as that of the dissolvable member without the plurality of apertures.

22. The device of claim 20, wherein the dissolvable member with the plurality of apertures is the same as that of the dissolvable member without the plurality of apertures.

23. The device of claim 19, wherein said first dry ingredient comprises dry sodium chlorite.

24. The device of claim 20, wherein said first dry ingredient comprises dry sodium chlorite.

25. The device of claim 21, wherein said first dry ingredient comprises dry sodium chlorite.

26. The device of claim 22, wherein said first dry ingredient comprises dry sodium chlorite.

27. The device of claim 23, wherein the first water permeable, cellulose material is selected from the group consisting of cloth and sponge and the second water permeable, cellulose material is selected from the group consisting of cloth and sponge.

28. A kit for using the device of claim 19, including a container for receiving the device of claim 19, said container being sized to receive a pre-determined amount of a liquid catalyst for facilitating a reaction between said first dry ingredient and said second dry ingredient within said packet.

29. A kit for using the device of claim 20, including a container for receiving the device of claim 20, said container being sized to receive a pre-determined amount of a liquid catalyst for facilitating a reaction between said first dry ingredient and said second dry ingredient within said packet.

30. A kit for using the device of claim 21, including a container for receiving the device of claim 21, said container being sized to receive a pre-determined amount of a liquid catalyst for facilitating a reaction between said first dry ingredient and said second dry ingredient within said packet.

31. A kit for using the device of claim 22, including a container for receiving the device of claim 22, said container being sized to receive a pre-determined amount of a liquid catalyst for facilitating a reaction between said first dry ingredient and said second dry ingredient within said packet.

32. A kit for using the device of claim 23, including a container for receiving the device of claim 23, said container being sized to receive a pre-determined amount of a liquid catalyst for facilitating a reaction between said first dry ingredient and said second dry ingredient within said packet.

33. A kit for using the device of claim 24, including a container for receiving the device of claim 24, said container being sized to receive a pre-determined amount of a liquid catalyst for facilitating a reaction between said first dry ingredient and said second dry ingredient within said packet.

34. A kit for using the device of claim 25, including a container for receiving the device of claim 25, said container being sized to receive a pre-determined amount of a liquid catalyst for facilitating a reaction between said first dry ingredient and said second dry ingredient within said packet.

35. A kit for using the device of claim 26, including a container for receiving the device of claim 26, said container being sized to receive a pre-determined amount of a liquid catalyst for facilitating a reaction between said first dry ingredient and said second dry ingredient within said packet.

36. A kit for using the device of claim 27, including a container for receiving the device of claim 27, said container being sized to receive a pre-determined amount of a liquid catalyst for facilitating a reaction between said first dry ingredient and said second dry ingredient within said packet.
